(19) United States
(12) Patent Application Publication
(10) Pub. No.: US 2006/0293381 A1
(43) Pub. Date: Dec. 28, 2006

(54) Fungicidal effect by regulating signal transduction pathways

(76) Inventors: Kaihei Kojima, Durham, NC (US); Joseph Heitman, Durham, NC (US); Yong-Sun Bahn, Seoul (KR)

Correspondence Address: MYERS BIGEL SIBLEY & SAJOVEC, PO BOX 37428, RALEIGH, NC, 27627, US

(21) Appl. No.: 11/472,856

(22) Filed: Jun. 22, 2006

Related U.S. Application Data
(60) Provisional application No. 60/693,203, filed on Jun. 23, 2005.

(51) Int. Cl. A61K 31/4025

(52) U.S. Cl. 514/422

(57) ABSTRACT

The present invention concerns methods of treating fungal infections and methods of screening compounds for activity in treating fungal infections. Methods of the invention include using an active compound such as fludioxonil to treat a Cryptococcus neoformans infection. Also included are methods and pharmaceutical compositions useful for treating fungal infections using a Hog1 activator such as fludioxonil and a calcineurin inhibitor in combination.

### RELATED APPLICATIONS

This application is related to and claims the benefit of U.S. Provisional Application No. 60/693,203 filed Jun. 23, 2005, which is incorporated by reference herein in its entirety.

### GOVERNMENT SUPPORT

This invention was made with Government support under grant number AI50438 from the NIAID. The United States Government has certain rights to this invention.

### FIELD OF THE INVENTION

The present invention concerns methods of treating fungal infections and methods of screening compounds for activity in treating fungal infections.

### BACKGROUND OF THE INVENTION

Pathogenic fungi have emerged as an increasing threat to both public health and the food industry. Proper treatments for limiting pathogenic fungal infection in both the natural environment and the human host are therefore important.

Fludioxonil (4-(2,2-difluoro-1,3-benzodioxol-4-yl)pyrrole-3-carbonitrile) is a phenylpyrrole fungicide derived from the antibiotic pyrrolnitrin. Fludioxonil is used as a fungicide to control a variety of important plant-pathogenic fungi such as Botrytis cinerea. Fludioxonil is a unique fungicide in that it acts through disrupting a signal transduction pathway. This is in contrast to most common fungicidal actions that are based on inhibitory effects on the biosynthesis of cellular components such as amino acids, nucleotides, lipids, and polysaccharides in fungi.

Understanding how a chemical disturbs fungal signaling pathways presents many other targets for the inhibition of fungal growth. In a model filamentous fungus, Neurospora crassa, mutants lacking the HOG1 mitogen activated protein kinase (MAPK) gene, OS-2, show osmosensitivity and resistance to fludioxonil.

Cryptococcus neoformans is a basidiomycetous opportunistic human fungal pathogen that infects the central nervous system of immunocompromised patients, causing life threatening meningoencephalitis. Cryptococcosis is one of the most common fungal infections diagnosed in AIDS patients, particularly in regions where antifungal drugs such as amphotericin B and fluconazole are not readily available. However, amphotericin B has a number of adverse side effects and fluconazole exhibits only fungistatic activity. Furthermore, mutants resistant to these drugs are emerging in Candida species and C. neoformans. Therefore, it has become an important issue to develop new antifungal agents that are fungicidal, less toxic, and employ different mechanisms of action for use in combination drug therapies.

### SUMMARY OF THE INVENTION

By investigating fungal signal transduction we discovered three different signaling pathways that are involved in sensitivity and resistance of C. neoformans to fludioxonil. (K. Kojima, et al. Microbiology (2006), 152:591-604, all of which is herein incorporated by reference.) We found that the Hog1 MAPK pathway promotes sensitivity to fludioxonil in C. neoformans, whereas the calcineurin and Mpk1 MAPK pathways mediate resistance to fludioxonil. Furthermore, simultaneous perturbation of the Hog1 and calcineurin pathways by combined treatment with fludioxonil and FK506 inhibits the growth of the pathogen even more effectively than fludioxonil alone.

A first aspect of the present invention is a method of treating a fungal infection in a subject in need thereof, comprising administering said subject a treatment effective amount of an active compound such as fludioxonil, an analog thereof, or a pharmaceutically acceptable salt or prodrug thereof.

A second aspect of the present invention is a pharmaceutical composition useful for treating cryptococcosis comprising an active agent in a pharmaceutically acceptable carrier; wherein said active agent is a Hog1 activator such as fludioxonil, an analog thereof, or a pharmaceutically acceptable salt or prodrug thereof.

A third aspect of the invention is a method of treating a fungal infection (e.g., cryptococcosis) in a subject in need thereof, comprising administering said subject, in combination, a Hog1 activator and a calcineurin inhibitor. In some embodiments the combination is a synergistic combination; in some embodiments the calcineurin inhibitor is administered in an amount effective to enhance the efficacy of the calcineurin inhibitor.

A fourth aspect of the invention is a pharmaceutical composition useful for treating a fungal infection comprising, in a pharmaceutically acceptable carrier, a Hog1 activator and a calcineurin inhibitor.

A still further aspect of the present invention is the use of an active agent (Hog1 activator or calcineurin inhibitor) as described above for the preparation of a medicament for the treatment of a disorder as described above.

### BRIEF DESCRIPTION OF THE DRAWINGS

FIG. 1: [A] Treatment of multiple strains of C. neoformans serotype A H99 with fludioxonil (1 ug ml⁻¹ and 10 ug ml⁻¹), calcineurin inhibitor FK506, and FK506+Fludioxonil (1 ug ml⁻¹). [B] Measurement of cell density of multiple strains of C. neoformans representing relative cell growth after incubation of C. neoformans in culture for 72 hours in various fludioxonil concentrations. The following serotype A strains were used for this assay: WT (H99) (♦), and cna1Δ (▴), hog1Δ (□) and cna1Δ hog1Δ (∘) mutant strains. [C] Measurement of WT H99, hog1Δ, and cna1Δ mutants grown for 48 hours exposed to disks containing 10 ug (Fludiox₁₀), 50 ug (Fludiox₅₀), or 100 ug (Fludiox₁₀₀) of fludioxonil; 2 ug (FK506₂) or 20 ug (FK506₂₀) of FK506; 5 ul of 100% ethanol (ETOH) and dimethyl sulfoxide (DMSO).

FIG. 2: Differential fungicidal sensitivity between C. neoformans serotype A strain H99 and serotype D strain JEC21. WT indicates wildtype cells; hog1 indicates a mutant Hog1 gene hog1Δ. S. cerevisiae is used as a control.

FIG. 3: Measurements of Hog1 MAPK activation by rapid dephosphorylation in response to fludioxonil in C. neoformans. Measurements of phosphorylated Hog1 (P-Hog1) and unphosphorylated Hog1 (Hog1) are shown in C. neoformans serotype A strain H99 (H99 WT), H99 cna1Δ, serotype D strain JEC21 (JEC21 WT), and S. cerevisiae. The dual phosphorylation status of Hog1 (T171 and Y173) was monitored using antibody specific for dual phosphorylation of p38 MAPK (P-Hog1). The same blot was stripped and then probed with polyclonal anti-Hog1 antibody as a loading control (Hog1).

FIG. 4: Measurements of fludioxonil sensitivity and Hog1 phosphorylation patterns in response to fludioxonil in various clinical and environmental serotype A [A] and serotype D [B] isolates. Measurements of phosphorylated Hog1 (P-Hog1) and unphosphorylated Hog1 (Hog1) were done using Western Blot analysis.

FIG. 5: Measurements of fludioxonil sensitivity in various strains of C. neoformans.

FIG. 6: [A] Measurements of morphological changes in response to fludioxonil treatment (10 ug ml⁻¹ for 48 hours) in serotype A H99 WT [A-b], cna1Δ [A-c], and hog1Δ [A-d]. As a control, serotype A H99 WT was grown without fludioxonil [A-a]. Cells were then observed by microscopy (bar=20 um). [B] Measurements of glycerol content in cell extracts in response to fludioxonil treatment in serotype A WT strain H99, cna1Δ, hog1Δ, and the serotype D WT strain JEC21. Cells were grown to mid-exponential phase and then incubated in 10 ug fludioxonil ml⁻¹ for the time indicated. Glycerol content in cell extracts was measured by a UV-glycerol assay procedure and normalized to dry cell weight. Two individual experiments were performed and standard deviations are presented as error bars.

FIG. 7: Treatment of H99 WT and multiple mutant strains of C. neoformans with cell wall defects with fludioxonil and sorbitol+fludioxonil for 48 hours

FIG. 8: Schematic diagram of pathways mediating antifungal effects on C. neoformans. Fludioxonil treatment activates the HOG pathway by rapid dephosphorylation of the Hog1 MAPK in the majority of C. neoformans strains, in which Hog1 is phosphorylated under normal conditions. Hog1 activation contributes to intracellular glycerol accumulation, causing cell swelling by rapid water influx and perturbing cell surface integrity, which may result in cell lysis or cytokinesis defects. Calcineurin and Mpk1 MAPK pathways independently contribute to fludioxonil resistance by promoting cell wall integrity.

### DETAILED DESCRIPTION OF THE INVENTION

The term “treat” as used herein refers to any type of treatment that imparts a benefit to a patient afflicted with a disease, including improvement in the condition of the patient (e.g., in one or more symptoms), delay in the progression of the disease, etc.

The term “pharmaceutically acceptable” as used herein means that the compound or composition is suitable for administration to a subject to achieve the treatments described herein, without unduly deleterious side effects in light of the severity of the disease and necessity of the treatment.

The phrases “concurrent administration,” “administration in combination,” “simultaneous administration” or “administered simultaneously” as used herein, interchangeably mean that the compounds are administered at the same point in time or immediately following one another. In the latter case, the two compounds are administered at times sufficiently close that the results observed are indistinguishable from those achieved when the compounds are administered at the same point in time.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention.

“Fungal infections” that may be treated by the present invention include any fungal infection of an animal subject, including but not limted to those caused by pathogens such as Cryptococcus spp., Candida spp., Aspergillus spp., Histoplasma spp., Coccidioides spp., Paracoccidioides spp. Blastomyces spp., Fusarium spp., Sporothrix spp., Trichosporon spp., Rhizopus spp., Pseudallescheria spp. dermatophytes, Paeciliomyces spp., Alternaria spp., Curvularia spp., Exophiala spp., Wangiella spp., Penicillium spp., Saccharomyces spp., Dematiaceous fungi and Pneumocystis carinii (See, e.g., U.S. Pat. No. RE38,984 to Abruzzo et al.). Thus examples of fungal infections include superficial mycoses such as ringworm, tinea, athlete's foot, toe-nail fungus and thrush, subcutaneous mycoses, and systemic mycoses (including primary and opportunistic) such as histoplasmosis, aspergillosis, candidosis, cryptococcosis, and pneumocystis.

Cryptococcus neoformans (C. neoformans) (a fungi of the Sporidiobolaceae family), as used herein includes all serotypes (A, B, C and D) thereof and all variants (e.g., var. neoformans and var. gattii) thereof. Cryptococcosis is the disease caused by the infection of an animal with C. neoformans.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

The disclosures of all United States patents cited herein are incorporated by reference herein in their entirety.

1. Active Compounds.

Active compounds (Hog1 activators) useful for carrying out the present invention include, in general, fludioxonil or analogs thereof, or difluorobenzodioxyl cyanopyrrole compounds or analogs thereof. Numerous such compounds are known, and examples are described in U.S. Pat. No. 4,705,800 to Nyyfeler et al. (assigned to Ciba-Geigy Corp); and in U.S. Pat. Nos. 4,925,840; 5,250,557; 5,496,848; 5,514,816; 6,080,749; 6,306,850; 6,503,904; 6,730,312.

Thus in some embodiments the compounds of this invention have the general formula I 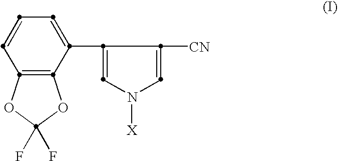
wherein X has the following meanings:

A: hydrogen or CO—R₁, wherein R₁ is C₁-C₆alkyl which is unsubstituted or substituted by halogen or C₁-C₃alkoxy; or is C₃-C₆alkenyl, C₃-C₆alkynyl, or C₁-C₆alkoxy which is unsubstituted or substituted by halogen or C₁-C₃alkoxy; or is C₃-C₆alkenyloxy, C₃-C₆cycloalkyl or tetrahydrofur-2-yl;

B: S—R₂, wherein R₂ is C₁-C₃haloalkyl;

C: CH(Y)R₃, wherein R₃ is hydrogen or C₁-C₈haloalkyl and Y is hydroxy, halogen or OC(O)R₄, wherein R₄ is C₁-C₈alkyl, C₁-C₈haloalkyl, C₂-C₆alkenyl, tetrahydrofur-2-yl, tetrahydropyran-2-yl or C₁-C₆alkoxycarbonyl;

D: CH₂-Z, wherein Z is one of the groups 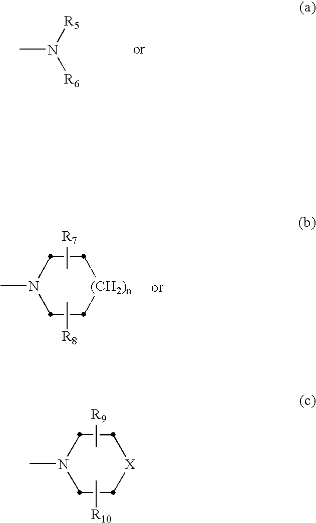
in which formulae each of R₅ and R₆ independently of the other is hydrogen, C₁-C₆alkyl which is unsubstituted or substituted by cyano or C₁-C₆alkoxycarbonyl; or is C₃-C₆alkenyl, C₃-C₆alkynyl, C₃-C₇cycloalkyl, or phenyl which is unsubstituted or substitued by halogen, C₁-C₆alkyl, C₁-C₆haloalkyl and/or C₁-C₆alkoxy, with the proviso that only R₅ or R₆ may be hydrogen; each of R₇ and R₈ independently of the other is hydrogen, C₁-C₆alkyl or C₁-C₆alkoxycarbonyl, or both together form a fused aromatic ring; each of R₉ and R₁₀ independently of the other is hydrogen, C₁-C₆alkyl or C₁-C₆alkoxycarbonyl; and X is oxygen, sulfur, 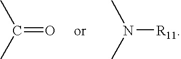
wherein R₁₁ is hydrogen, C₁-C₆alkyl, formyl, C₁-C₆alkanoyl or C₁-C₆alkoxycarbonyl; and n is 0 or 1.

Depending on the number of indicated carbon atoms, alkyl by itself or as moiety of another substituent will be understood as meaning for example the following groups: methyl, ethyl, propyl, butyl, pentyl, hexyl etc. and the isomers thereof, e.g. isopropyl, isobutyl, tert-butyl, isopentyl etc. Haloalkyl is a mono- to perhalogenated alkyl substituent, e.g. CH₂Cl, CHCl₂, CCl₃, CH₂Br, CHBr₂, CBr₃, CH₂F, CHF₂, CF₃, CCl₂F, CCl₂—CHCl₂, CH₂ CH₂F, Cl₃ etc. Throughout this specification, halogen will be understood as meaning fluorine, chlorine, bromine or iodine, with fluorine, chlorine or bromine being preferred. C₃-C₆Alkenyl is an unsaturated, aliphatic radical containing one or more double bonds, e.g. 1-propenyl, allyl, 1-butenyl, 2-butenyl, 3-butenyl, CH₃ CH═CHCH═CH-etc. Alkynyl will be understood as meaning unsaturated, aliphatic radicals containing a maximum of 6 carbon atoms, e.g. propargyl, 2-butynyl, 3-butynyl etc.

Under normal conditions the compounds of formula I are stable oils, resins or mainly crystalline solids which are distinguished by extremely valuable microbicidal properties. They can be used for example in agriculture or related fields preventively or curatively for controlling phytopathogenic microorganisms. The compounds of formula I are distinguished by a very good fungicidal activity in wide ranges of concentrations and their use poses no problems.

Compounds of formula I which are preferred on account of their pronounced microbicidal properties are those containing as X the following substituents or combinations of these substituents: hydrogen or CO—R₁, wherein R₁ is C₁-C₆alkyl which is unsubstituted or substituted by halogen or C₁-C₃alkoxy; or is C₃-C₆alkenyl, C₃-C₆alkynyl, or C₁-C₆alkoxy which is unsubstituted or substituted by halogen or C₁-C₃alkoxy; or is C₃-C₆alkenyloxy, C₃-C₆cycloalkyl or tetrahydrofur-2-yl.

Among the compounds of formula I which carry combinations of substituents defined in the above group, those compounds are particularly preferred wherein X has the following meanings: hydrogen or CO—R₁, wherein R₁ is C₁-C₄alkyl which is unsubstituted or substituted by chlorine, bromine or C₁-C₃alkoxy; or is C₃-C₄alkenyl, C₃-C₄alkynyl, or C₁-C₄alkoxy which is unsubstituted or substituted by chlorine, bromine or C₁-C₃alkoxy; or is C₃-C₄alkenyloxy, C₃-C₆cycloalkyl or tetrahydrofur-2-yl. See for instance, U.S. Pat. No. 4,705,800, which is herein incorporated by reference.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine; and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Prodrugs are to compounds that are rapidly transformed in vivo to yield the parent active compound of the above, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

2. Calcineurin Inhibitors.

In some embodiments, the subject is preferably also administered a calcineurin inhibitor. Such compounds are also “active agents” as used herein. Calcineurin inhibitors are known and described in, for example, U.S. Pat. Nos. 6,686,450; 6,492,325; 6,046,005; 5,807,693; 5,774,354; 5,723,436; and 5,629,163; and in U.S. Patent Applications Nos. 20050008640; 20040224876; 20040091477; 20040033941; 20030045679; and 20020019344. Specific examples include, but are not limited to, cyclosporin A, tacrolimus, FK506, ascomycin, pimecrolimus, and ISAtx247.

The calcineurin inhibitor and the Hog1 activator may be administered separately or combined together in a common pharmaceutically acceptable carrier.

Preferably the calcineurin inhibitor and the Hog1 activator are administered to the subject in a synergistic amount (e.g., the combined treatment effect of the two active compounds together is greater than the sum of the effect of the two active compounds when administered individually) and/or the calcineurin inhibitor may simply be administered in an amount effective to ehance the activity of the Hog1 activator in treating the disease or condition for which the Hog1 activator is being administered.

3. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound or active compounds. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unitdose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound as described above, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bistris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compounds described herein, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

4. Dosage and Routes of Administration.

The present invention may be utilized to treat fungal infections in both human and animal subjects. In some embodiments the subject is an immune impaired subject, such as a transplant patient undergoing immune suppression therapy, an HIV-1 patient or patient afflicted with AIDS, or a cat infected with FIV or FeLV.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 or 1 to about 50 or 100 mg/kg of each active compound may be used, with all weights being calculated based upon the weight of the active compound, including the cases where a salt is employed. A dosage from about 10 mg/kg to about 50 or 100 mg/kg of each active compound may be employed for oral administration. Typically, a dosage from about 0.5 mg/kg to 5 or 10 mg/kg of each active compound may be employed for intramuscular injection.

5. Screening for Additional Active Compounds.

The present invention further provides a method of screening a compound for fungicidal activity, for example against Cryptococcus neoformans. The method comprises contacting a fungal cell containing Hog1 to a test or candidate compound, and then detecting activation of Hog1 by said compound, activation of Hog1 indicating fungicidal activity of said compound. Activation may be as compared to Hog 1 activity in a corresponding control cell to which the test compound has not be contacted. In one embodiment the fungal cell is a Cryptococcus neoformans cell. In one embodiment, Hog1 activation is detected by detecting glycerol accumulation in the cell.

The present invention is explained in greater detail in the following non-limiting Examples.

### EXAMPLE 1

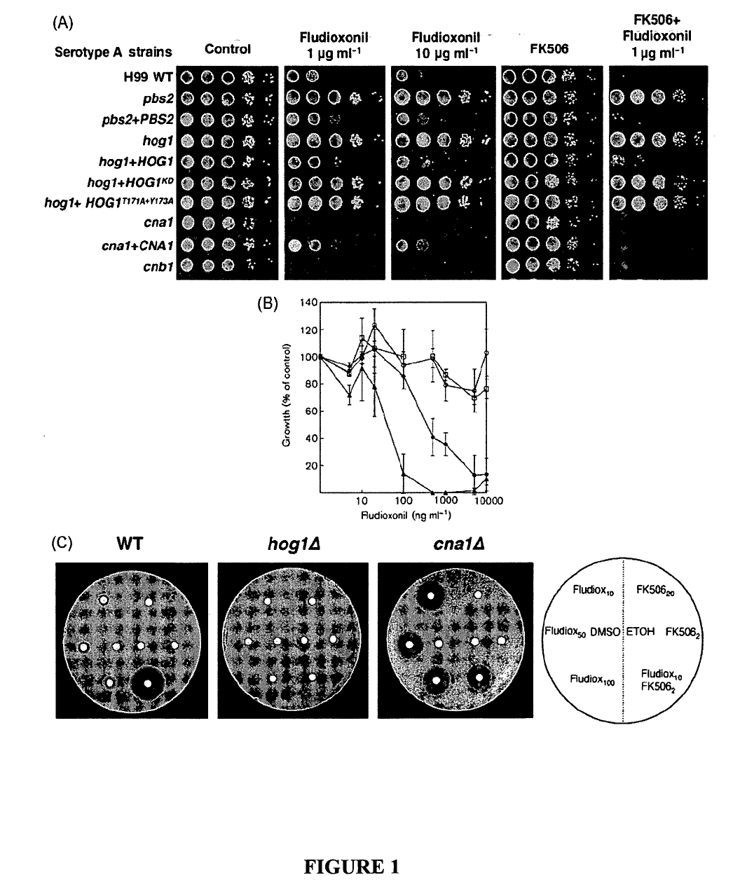

To investigate whether C. neoformans is sensitive to fludioxonil, fungal growth was tested on YPD agar containing the drug. Fludioxonil severely inhibited growth of the serotype A wild-type (WT) strain H99 in a dose dependent manner (FIG. 1A). To elucidate the role of the HOG pathway in fludioxonil sensitivity, we tested the sensitivity of hog1Δ and pbs2Δ mutants that had been constructed before (Y S Bahn et al., Mol. Biol. Cell. (2005) 16: 2285-2300). Both mutants exhibited complete resistance to fludioxonil, indicating that the Hog1 pathway is involved in fludioxonil sensitivity of C. neoformans (FIG. 1A). To examine whether phosphorylation and kinase activity of Hog1 MAPK are required to confer fludioxonil sensitivity, we tested the sensitivity of cells expressing site-directed mutants of Hog1 at the phosphorylation sites (hog1+HOG1^{T171A+Y173A}) or the catalytic site (hog1+HOG1^{K49S+K50N}) (FIG. 1A). These Hog1 mutants were as resistant to fludioxonil as the hog1Δ mutant, indicating that Pbs2-dependent phosphorylation and catalytic activation of the Hog1 MAPK are prerequisites for fludioxonil sensitivity (FIG. 1A).

We then tested whether calcineurin is also involved in resistance to fludioxonil. For this purpose, we deleted the genes encoding the calcineurin catalytic (CNA1) or regulatory subunit (CNB1) in the H99 background with dominant selectable markers. The cna1Δ and cnb1Δ mutants exhibited hypersensitivity to fludioxonil, indicating that calcineurin promotes resistance to fludioxonil in C. neoformans (FIG. 1A). We also tested whether a synergistic fungicidal effect would be observed with concomitant exposure to fludioxonil and the calcineurin inhibitor FK506. FK506 greatly enhanced growth inhibition when combined with fludioxonil, but had no effect on cell growth by itself (FIG. 1A), strongly suggesting a synergistic fungicidal effect between the two drugs. Interestingly, a cna1Δ hog1Δ double mutant still exhibited complete resistance to fludioxonil (FIG. 1B), indicating that calcineurin-dependent fludioxonil resistance is also mediated directly or indirectly by the Hog1 MAPK.

To quantitatively measure fludioxonil sensitivity, we performed drug susceptibility assays according to NCCLS criteria using a range of fludioxonil concentrations (5 ng ml⁻¹ to 10 ug ml⁻¹) to determine the minimum inhibitory concentration (MIC). In this assay, the MIC₈₀ of fludioxonil for the WT strain was <5 ug ml⁻¹ whereas the MIC₈₀ for the cna1Δ mutant was <100 ng ml⁻¹ (FIG. 1B and Table 1).

TABLE 1
Combination of fludioxonil and FK506 on C. neoformans

| Genotype | MIC₈₀ alone | | MIC₈₀ combined | FIC index |
|---|---|---|---|---|
| (C. neoformans | (μg ml⁻¹) | | (μg ml⁻¹)* | Fludiox/ |
| strain) | Fludiox | FK506 | Fludiox/FK506 | FK506 |
| Wild-type (H99) | <5 · 0 | >2 · 0 | ≦0 · 5/≦0 · 04 | 0 · 12 |
| cna1Δ (KK1) | <0 · 1 | >2 · 0 | — | — |
| hog1Δ (YSB64) | >10 · 0 | >2 · 0 | — | — |

*Combined MICs, expressed as [Fludioxonil]/[FK506], are the minimum concentrations of fludioxonil and FK506 that resulted in a fungicidal inhibition profiled when the two drugs were used in combination.

In contrast, hog1Δ and cna1Δ hog1Δ mutants exhibited a modest reduction of growth, but still showed robust resistance, even with 10 ug fludioxonil ml⁻¹ (FIG. 1B). Taken together, these findings indicate that sensitivity of C. neoformans to fludioxonil is oppositely regulated by the HOG and calcineurin pathways. Multiple signaling pathways thus mediate the action of fludioxonil against C. neoformans.

### EXAMPLE 2

To demonstrate the synergism between fludioxonil and FK506 in C. neoformans, we employed disk diffusion halo assays. Even a disk containing 100 ug fludioxonil exerted only modest growth inhibition of the WT strain H99. Growth of the WT strain was not inhibited by FK506 under these conditions. However, when fludioxonil was combined with FK506, the halo produced was completely clear and larger than the haloes produced by fludioxonil alone (FIG. 1C). To confirm that calcineurin was the target of the observed drug synergy with FK506, a cna1Δ mutant strain was also tested. When disks containing 10, 50, or 100 ug fludioxonil were placed over the cna1Δ strain, we observed large haloes similar to those of the wild-type strain exposed to fludioxonil in combination with FK506 (FIG. 1C). Fludioxonil and FK506 did not produce any haloes on the hog1Δ strain, which is consistent with the result that the hog1Δ mutant was resistant to medium containing fludioxonil and FK506 (FIGS. 1A and 1C). Additionally, the fractional inhibitory concentration (FIC) was calculated to determine the FIC index, of which a value <1·0 denotes a synergistic interaction. The calculated FIC index of fludioxonil is 0·12 with FK506, denoting a synergistic relationship between fludioxonil and FK506 (Table 1). These results indicate that FK506 participates in drug synergy with fludioxonil by inhibiting the calcineurin pathway.

To determine whether fludioxonil is fungicidal or fungistatic to C. neoformans, minimal fungicidal concentrations (MFCs) were investigated in accordance with the NCCLS criteria (Table 2).

TABLE 2
Combination of fludioxonil and FK506 on C. neoformans

| Genotype | MIC₈₀ alone | | MIC₈₀ combined | FIC index |
|---|---|---|---|---|
| (C. neoformans | (μg ml⁻¹) | | (μg ml⁻¹)* | Fludiox/ |
| strain) | Fludiox | FK506 | Fludiox/FK506 | FK506 |
| Wild-type (H99) | <5 · 0 | >2 · 0 | ≦0 · 5/≦0 · 04 | 0 · 12 |
| cna1Δ (KK1) | <0 · 1 | >2 · 0 | — | — |
| hog1Δ (YSB64) | >10 · 0 | >2 · 0 | — | — |

*Combined MICs, expressed as [Fludioxonil]/[FK506], are the minimum concentrations of fludioxonil and FK506 that resulted in a fungicidal inhibition profiled when the two drugs were used in combination.

Although fludioxonil dramatically inhibited growth of the WT strain in liquid medium (FIG. 1B), 10 ug fludioxonil ml⁻¹ did not produce an MFC against the WT strain, indicating that fludioxonil at <10 ug ml⁻¹ is not fungicidal against C. neoformans (Table 2). On the other hand, when fludioxonil was tested in combination with FK506, the MFC of fludioxonil was ≦0·5 ug ml⁻¹, indicating that the combination treatment of fludioxonil with FK506 has a fungicidal effect on the WT strain (Table 2). The MFC of fludioxonil for the cna1Δ mutant was ≦0·5 ug ml⁻¹, which is consistent with the MFC of fludioxonil in combination with FK506 against the WT strain.

### EXAMPLE 3

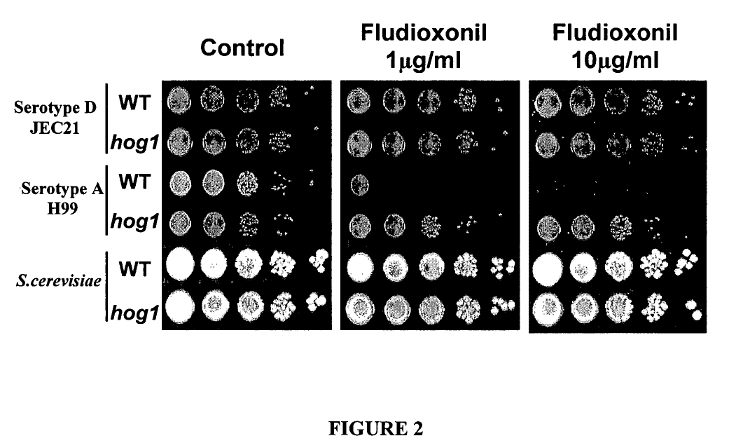

Two C. neoformans serotypes were tested for sensitivity to fludioxonil at 1 μg/ml and 10 μg/ml to determine whether fludioxonil sensitivity is differentially regulated between the two strains and if it is controlled by the HOG pathway. WT C. neoformans serotype A strain H99 exhibited sensitivity to fludioxonil at both concentrations (FIG. 2). WT C. neoformans serotype D strain JEC21 exhibited complete resistance to fludioxonil at both concentrations (FIG. 2). S. cerevisiae is resistant to fludioxonil and was used as a control. Thus differential sensitivity to fludioxonil was seen between the two WT serotypes. The hog1Δ mutation in the serotype D strain JEC21 was resistant to fludioxonil, similar to WT JEC21 (FIG. 2). The hog1Δ mutation in the serotype A strain H99 background, however, was resistant to fludioxonil, unlike WT H99 (FIG. 2). This indicates a critical role for the Hog1 pathway in fludioxonil sensitivity.

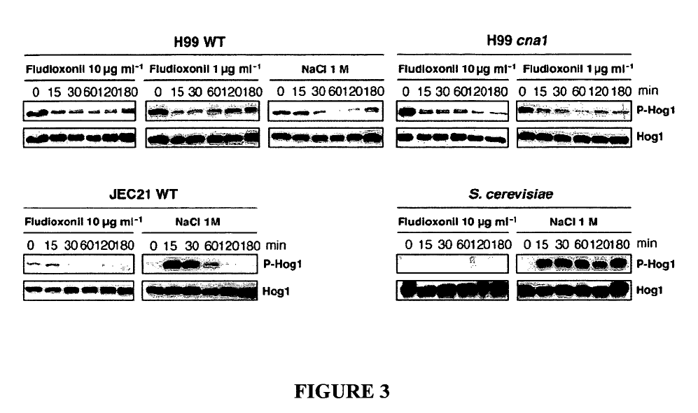

To determine how Hog1 is regulated in response to fludioxonil in C. neoformans, Hog1 phosphorylation patterns were monitored by Western Blot analysis in response to fludioxonil. When the serotype A strain H99 was exposed to 1 or 10 ug ml⁻¹ fludioxonil ml⁻¹, Hog1 was dephosphorylated within 15 minutes and its dephosphorylation status was maintained for 3 hours (FIG. 3). This regulatory pattern is quite similar to that of Hog1 in the H99 strain under osmotic stress (FIG. 3), indicating that fludioxonil activates the Hog1 pathway in WT H99 cells. On the other hand, in the serotype D strain JEC21 Hog1 was only slightly phosphorylated under normal conditions, only minimally further phosphorylated if at all after 15 minutes of exposure to fludioxonil, and subsequently maintained in an unphosphorylated state for up to 3 hours (FIG. 3). In contrast, Hog1 was rapidly phosphorylated in response to osmotic shock in strain JEC21. This shows that Hog1 is rapidly activated by dephosphorylation in response to fludioxonil in the drug-sensitive H99 strain, whereas Hog1 is only minimally activated, if at all, in the presence of fludioxonil in the resistant serotype D strain JEC21. Hog1 was also found to be completely inactive during exposure to fludioxonil in S. cerevisiae, which is also resistant to fludioxonil (FIG. 3).

The Hog1 phosphorylation pattern of the cna1Δ mutant was monitored in response to fludioxonil. The Hog1 phosphorylation pattern in the cna1Δ mutant exposed to 1 ug or 10 ug fludioxonil ml⁻¹ was almost identical to that observed in the wild-type strain in response to fludioxonil (FIG. 3). These data indicate that the calcineurin pathway promotes fludioxonil resistance, but does not directly regulate Hog1 phosphorylation or activation.

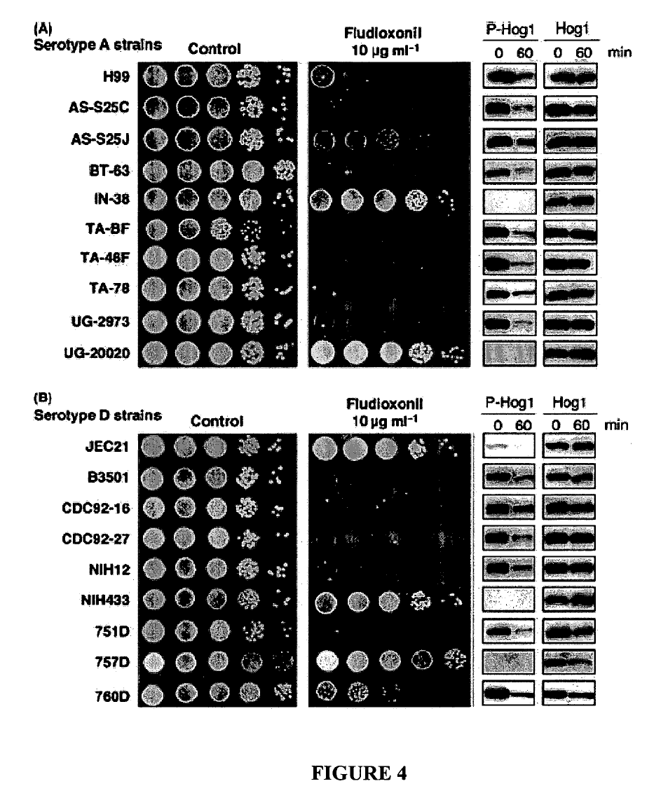

To determine whether the differential fludioxonil sensitivity observed between the serotype A strain H99 and the serotype D strain JEC21 results from serotype- or strain-specific differences, fludioxonil sensitivity in multiple serotype A and D clinical and environmental strains was investigated. The Hog1 phosphorylation pattern after a 1 hour exposure to fludioxonil was monitored. A majority of C. neoformans strains (8 of 10 serotype A and 6 of 9 serotype D strains) were found to be sensitive to fludioxonil, and in these strains Hog1 was regulated in a manner similar to that of the H99 strain (FIG. 4A and 4B). Two serotype A strains (IN-38 and UG-20020), and three serotype D strains (NIH433, JEC21, MMRL757) exhibited clear resistance to fludioxonil (FIGS. 4A and 4B). In the resistant strains, the Hog1 phosphorylation signal was almost undetectable under normal conditions, and this dephosphorylated state persisted after 1 hour incubation with fludioxonil, indicating that Hog1 is not activated in response to fludioxonil. Taken together, these data demonstrate that fludioxonil exerts a fungicidal effect via activation of the Hog1 pathway in a majority of C. neoformans strains.

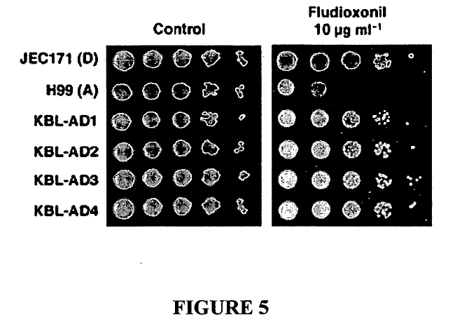

To investigate whether fludioxonil sensitivity is a dominant or recessive phenotype, the fludioxonil sensitivity of AD hybrid strains, which were laboratory generated by crossing between strains JEC171 (ade2 lys2) and H99 (ura5) (K B Lengeler et al. Infect. Immun. (2001) 69:115-122) was monitored. The parental control serotype A H99 (ura5) and serotype D JEC171 (ade2 lys2) strains exhibited sensitivity and resistance to fludioxonil, respectively (FIG. 5). All of 12 independently derived AD hybrid strains exhibited resistance similar to the parental strain JEC171, indicating that fludioxonil sensitivity is a recessive phenotype.

### EXAMPLE 4

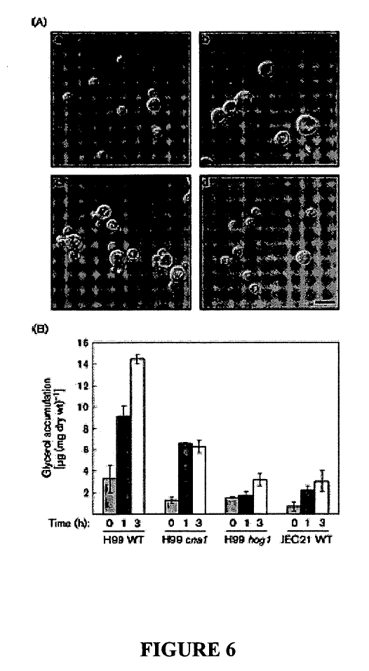

These data indicate that fludioxonil exhibits its fungicidal effect through the activation of Hog1. We microscopically observed cells after exposure to fludioxonil. In the WT some cells were swollen, and interestingly were often attached to each other, indicating a defect in cytokinesis during cell division (FIGS. 6A and 6B). Although the cna1Δ mutant strain exhibited a cytokinesis defect without fludioxonil, when this mutant strain was treated with fludioxonil, the cells exhibited an even more severe cytokinesis defect. On the other hand, a majority of hog1Δ mutant cells exhibited no swollen morphology or defects in cell division. As expected, the morphology of JEC21 cells was not affected by fludioxonil. These results demonstrate that fludioxonil-mediated cell growth inhibition is accompanied by defects in cell morphology and cell cycle that are dependent on integrity of the Hog1 pathway.

We measured the glycerol content in C. neoformans after fludioxonil treatment for 1 and 3 hours (FIG. 6B). In the WT strain H99, the glycerol content increased after 1 hour exposure, and to an even greater extent after 3 hour incubation. In contrast, no increase in glycerol was observed in the hog1Δ mutant compared to the WT. These results indicate that fludioxonil treatment hyperactivates the Hog1 osmotic response pathway, which results in over accumulation of intracellular glycerol. Increased intracellular glycerol levels may trigger non-physiological levels of water influx into the cell, resulting in cell swelling and growth inhibition. In the cna1Δ mutant, intracellular glycerol content increased following 1 hour treatment with fludioxonil but accumulation levels at 3 hours were lower than those of the WT (FIG. 6B). Thus, the cna1Δ mutant does not maintain intracellular glycerol levels similar to the WT strain, and may release glycerol to the extracellular environment, possibly due to impaired cell wall integrity. Alternatively, the cna1Δ mutant cells could be rapidly killed by Hog1 activation prior to accumulating glycerol, because fludioxonil has a fungicidal effect on the cna1Δ mutant (Table 1). As expected, the resistant strain JEC21 accumulated little or no glycerol after treatment with fludioxonil compared to the H99WT or cna1Δ mutant strains (FIG. 6B), further showing that Hog1 is not activated in strain JEC21 in response to fludioxonil (FIG. 3).

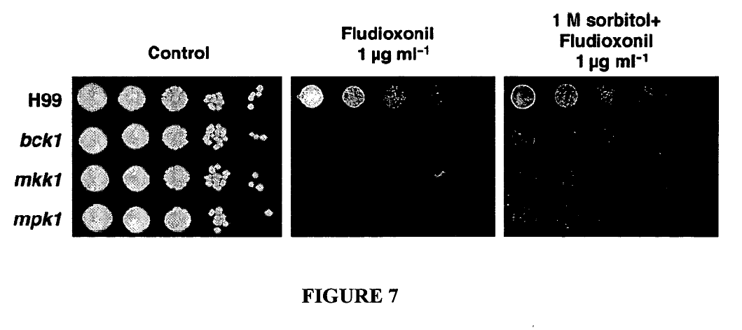

To test whether general defects in cell wall integrity result in hypersensitivity to fludioxonil, we examined the fludioxonil sensitivity of a mutant lacking the MPK1MAPK gene, which is also known to regulate cell wall integrity in C. neoformans. The mpk1Δ mutant exhibited a growth defect at 37° C. and hypersensitivity to fludioxonil similar to that of the cna1Δ mutant. In addition, C. neoformans mutants lacking the highly conserved MKK1 and BCK1 genes, which encode a MAPK kinase (MAPKK) and a MAPKK kinase (MAPKKK), respectively, which function upstream of the Mpk1 MAPK, also showed hypersensitivity to fludioxonil (FIG. 7). Supplementation with 1M sorbitol as an osmotic stabilizer partially rescued the growth defect of the mpk1Δ, mkk1Δ, and bck1Δ mutants in response to fludioxonil treatment. These results further support models in which cell wall integrity promotes cell viability in the presence of fludioxonil.

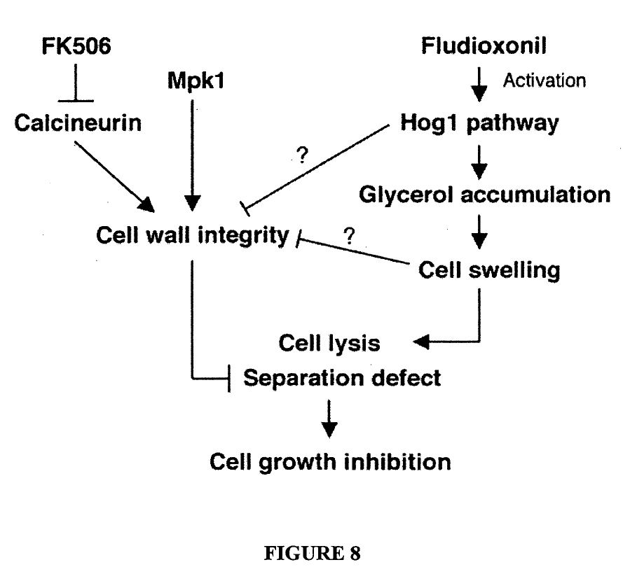

Our findings thus demonstrate that the phenylpyrrole drug fludioxonil exerts an antifungal activity against the basidiomycetous human fungal pathogen C. neoformans. Our findings further support a model where C. neoformans sensitivity to fludioxonil is not only positively controlled by the HOG pathway, but also negatively controlled by the calcineurin and Mpk1 MAPK pathways, which are involved in maintaining cell wall integrity (FIG. 8). Thus multiple different signaling pathways regulate the sensitivity and resistance of Cryptococcus neoformans to fludioxonil (FIG. 8). This discovery supports a novel treatment for cryptococcosis by simultaneously controlling two independent signaling pathways, the Hog1 MAPK and calcineurin pathways. A novel drug combination of fludioxonil and a calcineurin inhibitor exhibit synergistic fungicidal activity against C. neoformans, in contrast to the fungistatic activity by fludioxonil alone. Thus the simultaneous disturbance of these different signaling pathways inhibits the growth of fungus even more effectively than any one treatment alone. This expands options for the utility of existing antifungal drug classes, such as calcineurin inhibitors, by combination therapy with fludioxonil to exert synergistic antifungal effects.

The foregoing is illustrative of the present invention, and is not to be construed as limiting thereof. The invention is defined by the following claims, with equivalents of the claims to be included therein.

We claim:

1. A method of treating a Cryptococcus neoformans infection (or cryptococcosis) in a subject in need thereof, comprising administering an active agent to said subject in a treatment-effective amount; wherein said active agent is fludioxonil, an analog thereof, or a pharmaceutically acceptable salt or prodrug thereof.

2. The method of claim 1, wherein said subject is an immune impaired subject.

3. A pharmaceutical composition useful for treating cryptococcosis comprising an active agent in a pharmaceutically acceptable carrier; wherein said active agent is fludioxonil, an analog thereof, or a pharmaceutically acceptable salt or prodrug thereof.

4. A method of treating a fungal infection in a subject in need thereof, comprising administering said subject, in combination:
a. a Hog1 activator; and
b. a calcineurin inhibitor.

5. The method of claim 4, wherein said fungal infection is cryptococcosis.

6. The method of claim 4, wherein said Hog1 activator is is fludioxonil, an analog thereof, or a pharmaceutically acceptable salt or prodrug thereof.

7. The method of claim 4, wherein said calcineurin inhibitor is FK506 or a pharmaceutically acceptable salt or prodrug thereof.

8. The method of claim 4, wherein said Hog1 activator and said calcineurin inhibitor are administered to said subject in a synergistically effective amount.

9. A pharmaceutical composition useful for treating a fungal infection comprising, in a pharmaceutically acceptable carrier,
a. a Hog1 activator; and
b. a calcineurin inhibitor.

10. The composition of claim 9, wherein said Hog1 activator is is fludioxonil, an analog thereof, or a pharmaceutically acceptable salt or prodrug thereof.

11. The composition of claim 9, wherein said calcineurin inhibitor is FK506 or a pharmaceutically acceptable salt or prodrug thereof.

12. A method of screening a compound for fungicidal activity, comprising:
a. contacting a fungal cell containing Hog1 to said compound; and then
b. detecting activation of Hog1 by said compound, activation of Hog1 indicating fungicidal activity of said compound.

13. The method of claim 12, wherein said fungal cell is a Cryptococcus neoformans cell.

14. The method of claim 12, wherein said Hog1 activation is detected by detecting glycerol accumulation in said cell.
